(19) United States
(12) Patent Application Publication
(10) Pub. No.: US 2013/0096415 A1
(43) Pub. Date: Apr. 18, 2013

(54) METHOD TO DETERMINE SODIUM VALUES DESCRIBING THE CONTENT OF 23NA+, AND LOCAL COIL FOR USE IN SUCH A METHOD

(71) Applicants: Jan Ruff, Muenchen (DE); Jens Titze, Erlangen (DE); Peter Linz, Hemhofen (DE); Thoralf Niendorf, Aachen (DE); Davide Santoro, Berlin (DE); Wolfgang Renz, Erlangen (DE); Alexander Cavallaro, Uttenreuth (DE); Michael Uder, Uttenreuth (DE)

(72) Inventors: Jan Ruff, Muenchen (DE); Jens Titze, Erlangen (DE); Peter Linz, Hemhofen (DE); Thoralf Niendorf, Aachen (DE); Davide Santoro, Berlin (DE); Wolfgang Renz, Erlangen (DE); Alexander Cavallaro, Uttenreuth (DE); Michael Uder, Uttenreuth (DE)

(21) Appl. No.: 13/644,947

(22) Filed: Oct. 4, 2012

(30) Foreign Application Priority Data
Oct. 7, 2011 (DE) 102011084182.2

(51) Int. Cl. A61B 5/145; A61B 5/00; A61B 5/055

(52) U.S. Cl. CPC A61B 5/14546; A61B 5/055; A61B 5/748; A61B 5/7246; A61B 5/7278; A61B 5/1451; A61B 5/443; A61B 5/489
USPC 600/410

(57) ABSTRACT

In a method to determine at least one sodium value describing the 23Na+ content in at least one region of interest in a target region in the body of a patient, at least one sodium image data set of the target region is acquired with a magnetic resonance imaging device using sodium-23 imaging, the sodium image data set including image data dependent on the occurrence of sodium. The at least one region of interest is defined for which the sodium value is to be determined in the sodium image data set. The sodium value is determined by comparison of the image data in the region of interest with reference image data of at least one subject with a defined 23Na+ content, the reference image data having been acquired with the same sequence. A local coil can be used to implement the method that has a phantom integrated therein that allows the sodium image data set and the reference image data to be acquired together.

### BACKGROUND OF THE INVENTION

1. Field of the Invention

The invention concerns a method to determine at least one sodium value describing the 23Na+ content in at least one region of interest in a target region in the body of a patient, in particular for at least one compartment, as well as a local coil that can be used in such a method.

Fields of application of the invention are medicine and the medical technology industry.

2. Description of the Prior Art

Na+ metabolism is closely associated with correct cell function in mammals. Na+ is the predominant cation of the extracellular space and, as an osmolyte, determines the water content of the extracellular space, and thus the “milieu interieure” of the cell environment. The constancy of the extracellular volume therefore appears to be closely associated with a constant extracellular Na+ content. The presently accepted doctrine has previously been based on three paradigms for the role of the Na+ metabolism in the maintenance of the milieu interieur:

(1) Na+ storage in the organism primarily occurs in the extracellular space and inevitably leads to extracellular water retention. In order to avoid an extracellular volume excess, the Na+ content of the body must be kept within the narrowest limits (55-60 mmol/kg moisture mass). The extracellular volume is determined primarily by the intracellular content of K+.

(2) In order to avoid fluid shifts between intra- and extracellular space, the content of intra- and extracellular osmolytes is the same (iso-osmolality). The cell membrane-bound Na+/K+ ATPase maintains the functional disequilibrium across the cell membrane in that it pumps Na+ and K+ out of or into the cell, counter to their chemical gradients, while consuming energy.

(3) The monitoring of the extracellular Na+ content—and therefore of the extracellular fluid volume—falls to the kidneys. The Na+ content of the body is predominantly regulated hormonally by steroid hormones with mineralocorticoid effect. A failure of this hormonally controlled monitoring the extracellular Na+ content leads to an increase of the extracellular volume, and therefore to a rise in blood pressure.

New findings have recently placed these paradigms of regulation of salt and water content into question. Contrary to the previous assumption, the body's Na+ content does not need to be kept within the narrowest limits in order to maintain the extracellular volume. Large quantities of Na+ can be absorbed into the body without accompanying fluid retention. This takes place via redistribution of body cations. Contrary to the previous assumption, given massive increase of the body's Na+ content, large quantities of Na+ are absorbed into the body's cells and are exchanged for intracellular K+, such that the sum of the effective osmolytes (and therefore the fluid volume in the body or in the organs) remains unchanged (osmotically neutral Na+/K+ exchange). Moreover, cations can be accumulated at negatively charged connective tissue matrices or intracellular structure molecules and thus lose their hydrophilic properties (osmotically inactive Na+ storage). Contrary to the previously valid doctrine, not only the kidney and their associated hormonal regulation systems but even cells of the body and extracellular connective tissue are accordingly in the position to regulate the extracellular fluid volume (and therefore the blood pressure). This aspect of the extra-renal volume and blood pressure regulation offers new ways in detecting and treating Na+-associated health disorders since only the role of the kidneys and the volume-regulating hormone systems has previously been focused on in diagnostics and therapies, while changes to the distribution of the body's Na+ have not since been considered.

The fields of application in medicine that are directly derived from the concept of the extra-renal volume and blood pressure regulation primarily relate to patients with high blood pressure disorder, kidney function limitation (in particular dialysis patients), patients with edema formations (edema given cardiac and liver insufficiency, venous and lymph vessel illnesses, functional disruptions of the thyroid or idiopathic edema) and patients with reduced or increased extracellular Na+ concentration (hypo- or hypernatremia). Approximately 40 million people in Germany presently have sub-optimal blood pressure values, and 20 million people in Germany suffer from manifest high blood pressure illness. The cause of hypertonia is known in only 3 million people. Many of these patients appear to have an increased flow or effect of hormones with mineralocorticoid effect (for example Conn syndrome, Cushing syndrome, 10-OH-hydroxylase deficiency etc.). The strategy to detect arterial hypertonia is based on the classical pathophysiological concepts of Na+ metabolism (measurement of the renal Na+ excretion, measurement of the plasma aldosterone/renin quotients in the blood, hormone suppression tests). However there presently exist no methods to detect the Na+ redistribution within the body given hypertonia disease. It is to be expected that a method to measure Na+ redistribution disruptions will deliver a significant contribution to the clarification of the cause of the present form of hypertonia.

The detection of the body's Na+ content in patients with renal insufficiency is similarly problematical. Approximately 80,000 people with renal insufficiency requiring dialysis presently live in Germany. For dialysis patients, the success of the reduction of the body's Na+ content within the scope of the dialysis treatment is estimated solely from the estimate of the extracellularly removed fluid quantity, via determination of the dialysis end weight. This previous procedure is based on the traditional understanding that a Na+ metabolism occurs nearly exclusively in the extracellular volume. Na+ redistribution in intracellular compartments or osmotically inactive compartments have thereby not been considered since. The extent of the body's Na+ increases has most probably previously been dramatically underestimated in dialysis for the lack of a measurement method to assess such Na+ redistribution disruptions. A method to assess the Na+ redistribution would thus offer a new tool for the clinical management of dialysis treatment.

The principle problem of assessing the Na+ metabolism has previously been that methods were used in the sense of a “black box” approach. Findings about changes of the absolute Na+ content relative to changes of the absolute water content in the body have previously been described exclusively by balancing the Na+ or, respectively, water take-up and excretion or, respectively, via isotope dilution. A view into the body to assess the distribution of the absolute Na+ content in the various compartments of the body (bones, connective tissue, muscles, internal organs, brain) has not previously taken place. In recent months, information has been made accessible regarding internal Na+ distribution in different experimental forms of arterial hypertonia and the body's Na+ excess, and the concept of extra-renal Na+ and volume homeostasis has been developed. Although the “view into the body” via dry ashing delivers precise and previously inaccessible information about electrolyte and water shifts underlying specific illnesses, it was naturally limited to a purely experimental approach with animals. Therefore, a non-invasive method had to be developed that also made shifts of the internal Na+ and water distribution measurable in people.

Consequently, there have previously been no known methods which can determine the sodium content (concretely the Na+ content) in different regions of the body or compartments of living mammals, in particular a patient. Nephrological and cardiological disruptions of the sodium balance in the human body could previously be estimated only via the blood count and urine measurements. However, these allow only insufficient information about the actual load state in the tissue.

### SUMMARY OF THE INVENTION

An object of the invention is to provide a method with which the Na+ content in various regions within the body—for example in the skin, the muscles, internal organs and/or nerve cells—can be determined non-invasively and in vivo.

To achieve this object, in a method of the aforementioned type the following steps are implemented according to the invention:
acquire at least one sodium image data set of the target region that includes image data describing the Na+ content, using sodium-23 imaging and a magnetic resonance sequence with a magnetic resonance device,
define the at least one region of interest for which the sodium value should be determined in the sodium image data set,
determine the sodium value by comparison of the image data in the region of interest with reference image data of at least one subject having a defined Na+ content, which reference image data were acquired with the same magnetic resonance sequence.

In this way it is possible for the first time to determine Na+ contents non-invasively and divided up according to body regions. From this an Na+ distribution can also be determined from which distribution disruptions can be concluded. A simple, non-invasive method for quantitative assessment of the Na+ metabolism is consequently provided to the clinician for the first time. For this it is concretely proposed to use 23Na MR imaging.

Until today, sodium-23 (²³Na⁺) magnetic resonance tomography (MRT) and MR spectroscopy have not been methods of routine clinical diagnostics. The main reasons are the comparably long measurement times given relatively low spatial resolution and the necessity of additional specific preamplifiers and transmission and reception coils suitable for 23Na+. In comparison to 1H+, the MR sensitivity for 23Na+ is lower by a factor of 10 to the 4th (10,000) due to the lower gyromagnetic ratio and the lower biological frequency. Due to the interactions of the quadrupolar moments of 23Na+ with the electrical field gradients in their direct environment, the T1 and T2 relaxation times are markedly shorter than given 1H+. The lower absolute sensitivity of 23Na+ can thus be partially compensated via the selection of short repetition times. In biological tissues, the transverse relaxation shows a bi-exponential curve with a slowly relaxing component T2_{slow} of approximately 20 ms at 3 T and a rapidly relaxing component T2_{fast} of approximately 2 ms. Different quadrupolar interactions in the various tissue compartments (intracellular space, extracellular space, blood) are the cause. The ratio of the amplitudes between slowly and rapidly relaxing portions varies depending on the tissue to be examined. Nevertheless, the complete separation of the intracellular and extracellular Na+ due to the different relaxation characteristic is critical since an additional sub-compartmentalization is primarily present in damaged cells within the intracellular space. This should be dealt with in detail in the following.

The water content of biological tissue can be measured with proton MRT (¹H⁺). If the transversal relaxation process is considered and local field inhomogeneities are corrected, the signal intensity of proton density-weighted exposures directly maps to the water content of the tissue. Alternatively, the water content can also be calculated from the ^{|}H⁺ peak of proton spectra. The combination of sodium-23 imaging and the typical proton imaging (1H+) enables the non-invasive determination of the Na+ and water content in various tissues, thus body regions of interest. Shifts of the internal Na+ distribution can thereby be measured.

It is noted that all steps of the measurement method according to the invention which determines physical/technical measurement data with the Na+ content (which measurement data can subsequently be diagnostically evaluated) can be automated, for example in a control device of the magnetic resonance device. In particular, this means that both the determination of the body regions and the determination of the sodium value can preferably be implemented automatically.

In a practical implementation, it can initially be provided to register the patient with the magnetic resonance device. For this purpose the patient is moved into the magnetic resonance device, for example “feet first supine”, with a local coil selected for the particular data acquisition having been arranged at the target region (a leg, for example). As is typical, the target region is placed in the isocenter (homogeneity region) of the device since homogeneity is important with regard to the imaging goal.

It can then be provided to initially acquire a localizer image by using a coil adapted to proton imaging (a whole-body coil, for example). Such a coil is known in the prior art. The acquisition of the sodium image data set or image data sets then follows.

To assess the measurement values from the regions of interest and their associations with the sodium content, the method according to the invention also proposes to use reference data in which it is known what sodium content (concretely which concentration, for example) they correspond to. However, it is should be considered that every body tissue has a time and amplitude response of the magnetic resonance signal that is different from a phantom forming the basis of the reference data, and that additional depth and mass effects would have to be taken into account. The sodium value that is ultimately determined in the method according to the invention is to be considered a relative value—consequently a method-specific value. However, it can be provided that the sodium value can be converted (under consideration of calibration data) into a value reflecting the absolute Na+ content, for example a value reflecting an absolute concentration. For example, in order to determine calibration data, measurement data obtained in a chemical analysis of tissues (in particular via ashing) can be considered via the absolute Na+ content of the tissue in its connection with image data of the same tissue that were previously acquired with the magnetic resonance sequence.

In an embodiment of the method according to the invention it can be provided that a phantom supplying the reference image data is acquired together with the target region.

In this embodiment of the present invention, the image data of the target region are consequently acquired simultaneously with the reference data using a phantom (as a subject or forming the at least one subject) to supply the reference image data is. Such a phantom (which, for example, can embody a sodium chloride solution of predetermined Na+ content or an agarose block of predetermined Na+ content) is placed near the patient (in particular near the target region) before the acquisition of the at least one sodium image data set begins.

In order to achieve this, a phantom is integrated into the local coil that is used to acquire the sodium image data set. Thus the phantom is already integrated into a local coil (which is placed within or adjacent to the target region) designed specifically for sodium-23 imaging, such that measurement data (here the reference image data) can be acquired. The integration is preferably directly below the placement area for the target region, or in a corresponding receptacle, for example terminating flush with the placement area, such that the target region and the phantom also lie as close together as possible in the arising sodium image data set and consequently have similar acquisition conditions.

However, it is also generally preferable, to acquire the sodium image data set, for the target region to be directly adjacent to the phantom, in particular on the phantom. As has been explained, similar acquisition conditions exist for the target region and the phantom. Moreover, it is also conceivable to provide the phantom with a step and/or bevel, wherein the higher region (which can likewise form a placement area) includes at least one material-defined Na+ content, such that image data of the target region which lie at the lower region of the phantom suitably lie in-plane with the reference data for comparison. The lower region of the phantom can include, for example, a material without sodium so that a good contrast is provided. An additional advantage of such a step is that the target region is arranged nearer to the coil elements of the local coil (at least in the lower region of the phantom), which increases the signal-to-noise ratio.

The fact that the target region is arranged adjacent to the phantom (in particular rests thereon) also enables at least one region of interest to be defined from its relative position to the phantom, in particular thus from the reference data that can be located in the sodium image data set. This preferably occurs automatically within the scope of an image evaluation; such a procedure has proven to be particularly suitable for the determination of the skin as a region of interest. The skin lies directly on the placement area of the phantom, in particular is consequently the first part visible from the target region after the phantom or its reference data, such that it can be located automatically. For example, in an embodiment first pixels of the target region on the phantom can be evaluated as skin. At this point it is thereby already apparent that the skin is quite thin, and due to the weak sodium signal the thickness of the skin most often already essentially corresponds to the voxel size, which is discussed in further detail in the following. Assuming that the material of the phantom body itself contains no sodium and delivers corresponding reference measurement data results in a reliable detection of the position of the skin, since then the best contrast is provided.

The phantom can appropriately have multiple containers and/or receptacles for materials of different Na+ content. In this way a manner of scales can be achieved so that the reference data deliver values for different Na+ contents. For example, regular intervals of the Na+ content can be used so that between which values an image datum lies can also be established optically (for example by comparison).

As mentioned, a sodium chloride solution can already be used as a material, consequently a fluid material in which the Na+ is dissolved and consequently exist so as to be freely movable. However, it is also possible to integrate the Na+ into a different material (for example a solid or a gel), wherein agarose can advantageously be used. The embedding of NaCl into 5% agarose in particular has the additional advantage that the T2 time is comparable with that of the skin, and thus that calibration errors are reduced. Such NaCl agaroses can be cast in prefabricated molds, for example. It is noted that it is in principle possible to also support a patient directly on the agarose, but with an open storage of the agaroses the problem exists that these can dry out quickly. Therefore, it is preferred to store the agaroses in a sealed container or a sealed receptacle so that they are consequently reusable.

As already indicated, it can be provided that at least four materials equidistant relative to the Na+ content are used so that ultimately a type of scale is provided. Here, for example, it is suggested to use materials with 10, 20, 30 and 40 mM NaCl or materials with 0, 20, 40 and 60 mM NaCl (mM stands for mmol/liter).

Overall, the phantom can be designed in various ways. For example, a base body with multiple receptacles for the multiple materials (for example empty, oblong, semi-cylindrical spaces that are then filled with the material—for example a sodium chloride solution) is also conceivable, whereupon the spaces are covered and externally sealed via a covering on a side of the base body. It is suggested to design the cover facing the patient (which consequently ideally offers a placement surface) to be thin, for example as a thin layer which can be formed by a membrane and/or a film. Such a thin layer is of less consequence with regard to partial volume effects.

In a further embodiment of the invention, the position of the materials—and therefore the reference image data—is determined in the sodium image data set via segmentation. A—preferably automatic—image evaluation can also be provided with regard to the various materials, in particular consequently a segmentation that locates the corresponding regions of suitable identical image data. Such methods are known in principle and facilitate the workflow during the implementation of the method according to the invention.

As mentioned, the magnetic resonance signal that is achieved in the sodium-23 imaging is rather weak, such that in general poorer spatial resolution is provided than given comparable proton imaging. The signal—and consequently also the spatial resolution—can be increased with rising basic magnetic field of the magnetic resonance device. It is consequently preferred to use a magnetic resonance device with a basic field strength of at least 3 Tesla (in particular at least 7 Tesla). For example, if 3 Tesla magnetic resonance devices have a good spatial resolution for proton imaging into the sub-millimeter range (consequently offer an excellent tissue contrast of the water-rich organs), 23Na magnetic resonance tomography delivers a resonance signal weaker by a factor of 10⁴ due to the lower concentration of sodium in the body in comparison to hydrogen and due to intrinsic factors of the nuclei. However, the high magnetic field strength according to the invention allows a usable spatial resolution even given the low magnetic resonance signals. Given a basic field strength of 3 Tesla using reasonable clinical measurement times, an in-plane resolution of 3 millimeters can be achieved, such that certain partial volume effects are provided, for example if the skin (which has a thickness of only approximately one millimeter) is considered as a region of interest. A field strength of 7 Tesla or more which markedly increases the spatial resolution, for example up to one millimeter in-plane (even to 0.5 millimeter in-plane, for example) with T1-weighting, is particularly preferred.

A gradient echo sequence (in particular with an echo time of 1.5 milliseconds or more) can be used to acquire the or at least one sodium image data set. Relative to conventional spin echo sequences of proton imaging, a gradient echo sequence has the advantage that the echo time (TE) can be chosen to be shorter. Since the magnetic resonance signal decays very quickly in sodium-23 imaging but a representation of sodium contents (in particular concentrations in tissue that is as close to reality as possible) is desired, each shortening of the echo time leads to stronger signals and to a reduction of an unwanted, environment-dependent T2 contrast as it is typically used in proton imaging.

As indicated, the magnetic resonance signal of 23Na essentially has two decay times. Physiological sodium chloride solutions with freely mobile Na+ ions have “long” time constants which, at 3 Tesla, lie in a range from 15-30 milliseconds, for example. For Na+ in body tissue, for example in muscle or in the skin, an additional fast component is added that is ascribed to molecular interactions and lies in the range from 0.5 to 8 milliseconds (again for 3 Tesla).

In an appropriate embodiment, in particular when differentiation should be made between freely mobile Na+ and bound Na+, it can be provided that a gradient echo sequence with more than one echo (in particular up to 12 echoes) is used. The option thus exists to develop up to 12 echoes after an excitation so that the proportion of bound sodium can be completely eliminated in practice. A sodium image data set is ultimately acquired that primarily relates to freely mobile Na+. If a sodium image data set now additionally exists, in particular one that was acquired with markedly even shorter echo times that shows the entire sodium content, a value of “bound” sodium can thus also be determined by calculating the difference, whereupon this will be discussed in detail later.

Within the scope of a gradient echo sequence, a T2-weighting is provided anyway in the acquisition of the sodium image data set. If a T1 weighting is additionally accepted, the in-plane resolution can be improved, for example up to 0.45 millimeters at 7 Tesla.

The (or at least one) sodium image data set can be acquired with a sequence with echo times less than one millisecond, in particular a radial sequence. Given such radial sequences that are basically known in the art, even markedly shorter echo times can be achieved than given gradient echo sequences, for example in the range from 0.1 to 0.5 milliseconds. A radial frequency is consequently particularly suitable in order to determine the total Na+ content within the regions of interest. It is therefore particularly advantageous to acquire a sodium image data set with a gradient echo sequence and an additional sodium data set with a radial sequence, wherein the sodium image data set acquired with the gradient echo sequence preferably has multiple echoes in order to suppress bound portions of sodium. Nevertheless, it is thus possible to also determine values for the bound sodium by calculating the difference. The latter is in turn connected with intracellularly stored sodium or sodium stored in another manner (as has already been explained), such that observations can also be made in this regard.

One possibility to differentiate between intracellular and extracellular Na+ would also be what is known as the shift reagents. The differentiation of intracellular and extracellular Na+ is possible with the aid of shift reagents. Shift reagents are negatively charged complex compounds of paramagnetic metal ions (thulium, for example) that form ion pair bonds with biological cations and therefore alter their direct magnetic environment. Cell membranes are not permeable to these shift reagents, such that the resonance frequency of the extracellular Na+ is shifted while that of the intracellular Na+ remains nearly unaffected. However, such shift reagents have not previously been permissible for clinical use.

In a further embodiment of the present invention, it can be provided that at least two (in particular four) sodium image data sets are acquired with the same sequence, the averaged image data of which are used for evaluation. Statistical errors can be minimized in this way.

A correction with regard to B1 inhomogeneities can be implemented for at least one sodium image data set, in particular given a field strength of at least 7 Tesla and use of a local coil matched to sodium-23 imaging. At high basic field strengths, the distance-dependent surface inhomogeneity of a local coil is disadvantageous for the Na+ quantification, which has the effect that near subjects in spin density-weighted exposures appear brighter and at higher resolution than subjects distant from coil elements of the local coil. This problem can be remedied by such a correction. In a further embodiment, a correction image data set of a subject having a homogenous Na+ content is acquired at the position of the target region with the same sequence as the sodium image data set to be corrected, as that sodium correction image data set is used for correction, in particular by an image point-by-image point correction of the sodium image data set by the correction image data set. For example, an agarose phantom can be used, and a longer measurement can also be implemented in order to further increase the precision of the correction image data set, possibly even with an averaging of multiple image data sets acquired over a longer time period (two hours, for example). As long as the same magnetic resonance sequence is used, such a correction image data set can even be used for multiple examinations, in particular different patients.

In principle, a more precise procedure with a B1 correction for each specific patient or each target region is conceivable, but it has been shown that a sufficiently precise correction is possible even using such a correction image data set of a subject, in particular a calibration phantom.

In a further embodiment of the present invention, at least one anatomy image data set showing the anatomy of the patient in the target region is acquired using hydrogen imaging with the magnetic resonance device with the target region that not being moved in comparison to the sodium image data set, and a segmentation of a region of interest is transferred from the anatomy image data set to the sodium image data set. It is consequently advantageous to also acquire an anatomy image data set in order to be able to more precisely determine the region of interest, which is possible to accomplish from the sodium image data set only in rare cases, for example in the case of the skin (as was described). For example, it is thus possible to locate musculature, organs and the like in the anatomy image data set via automatic, semi-automatic or manual segmentation methods, whereupon this segmentation can easily be transferred to the sodium image data set due to the same magnetic resonance device that is used and the unmoved target region, such that the image data that enter into the sodium value of a region of interest should consequently be selected in the manner of a mask in the sodium image data set.

In this context it is advantageous for blood vessel regions including water regions and/or visible blood vessels to be segmented in the anatomy image data set and excluded in the evaluation of the sodium image data set. It is thus possible to already exclude in advance regions in which a high sodium content is assumed anyway, which high sodium content could have an adulterating effect on the measurement in the region of interest. Moreover, such regions can in principle also be segmented in the sodium image data set where they are most conspicuous due to a very high image value.

As mentioned, it is advantageous to also consider the skin as a region of interest. This can be done automatically in a segmentation algorithm to determine the skin as a region of interest, wherein a region containing air or no sodium is monitored based on exceeding a threshold (in particular double the background noise). Such a segmentation is also possible in the sodium image data set itself since it has been shown that the skin frequently has a high sodium content. It is also possible to use a threshold amounting to ten times the background noise, for example. Because the skin is extremely thin (most often its extent encompasses one image point or one voxel of the sodium image data set), the skin can be assumed to be, for example, one image point (specifically one voxel) wide.

Various possibilities are possible to determine the sodium value from the image data. Within the scope of the present invention it is particularly preferable to implement a linear trend analysis using at least two reference image data sets related to different sodium contents to determine the sodium value. If image values that correspond to determined sodium contents are consequently known from the reference image data, a characteristic line can be determined that linearly connects these points. A corresponding sodium content can then be read out as a sodium value on such a characteristic line for an (averaged) image value measured in a region of interest. It has been shown that such an assumption leads to good results. The calculation method can be implemented and conducted easily.

As noted, the method according to the invention represents an extremely useful assistive means in the diagnosis of illnesses related to the sodium balance or, respectively, the sodium distribution. In addition, it assists in the fundamental research which concerns the causes and effects of such distribution disruptions. Consequently, the at least one sodium value can be evaluated with regard to a distribution disruption of Na+ in the body of the patient and/or a disruption of the absolute Na+ content in the body of the patient. Various application cases are thereby conceivable. For example, if Na+ contents and water contents in the human body are considered simultaneously, in the case of hyponatremia it can stand out that a higher—in particular too high—sodium content nevertheless exists in the body of a patient given the same water content. However, this is only one specific application case in which the method according to the invention can advantageously be used. In general, a number of additional fields of applications are thus conceivable, among which the following are examples. The sodium values obtained according to the invention can thus be evaluated with regard to
the detection of disruptions that, due to increased activity of mineralocorticoid hormones, accompany increased osmotically neutral Na+/K+ exchange or osmotically inactive Na+ storage or increase of the Na+ content in heart and skeletal musculature, brain, liver and other internal organs,
detection of disruptions that, due to reduced (increased) Na+ excretion on the part of the kidneys or increased (reduced) Na+ retention in the body, accompany osmotically neutral Na+/K+ exchange or osmotically inactive Na+ storage and increase (decrease) of the Na+ content in heart and skeletal musculature, brain, liver and other internal organs,
detection of disruptions that, due to medicinal or nervous constraint of the Na+/K+ ATPase, accompany osmotically neutral Na+/K+ exchange or osmotically inactive Na+ storage and increase of the Na+ content in heart and skeletal musculature, brain, liver and other internal organs (also suitable for localization diagnostics of benign or malignant tumors),
detection of forms of high arterial blood pressure that are to be ascribed to increased production or reduced decomposition of hormones with mineralocorticoid effect (aldosterone, cortisol and other steroid hormones with mineralocorticoid effect),
changes of the body Na+ content in patients with renal insufficiency, in particular in dialysis patients (reduced Na+ excretion, reduced Na+ storage),
detection of the disruption forming the basis of a hyponatremia, in that differentiation is made between absolute body Na+ loss (reduced Na+ excretion, reduced Na+ storage) or relative excess extracellular water,
detection of the disruption forming the basis of a hypernatremia, in that differentiation is made between absolute body Na+ excess (reduced Na+ excretion, increased Na+ storage) or relative excess extracellular water deficit,
detection of forms of high arterial blood pressure that accompany increased activity of the sympathetic nervous system or other nervous constraint of the Na+/K+ ATPase with osmotically neutral Na+/K+ exchange or osmotically inactive Na+ storage and increase of the Na+ content in heart and skeletal musculature, brain, liver and other internal organs,
detection of biological aging processes due to Na+ storage in the tissues,
detection of causes of high blood pressure illnesses,
detection of disruptions of the body's Na+ supply given renal insufficiency, in particular given renal insufficiency requiring dialysis,
disruptions of the body's Na+ supply given cardiac insufficiency,
disruptions of the body's Na+ supply given edema illnesses, in particular given edemas at the base of renal, cardiac, hepatic, thyroid, venous and lymph vessel illnesses, and given idiopathic edemas,
detection of the causes of hyponatremia and hypernatremia.

In a special embodiment of this further evaluation of the sodium value according to the invention, a time curve of at least one sodium value is considered in multiple measurements made at different points in time. For example, given patients with hypernatremia or hyponatremia the Na+ content can be considered and compared before and after a dialysis in order to be able to reliably diagnose these illnesses and the like. Even given other illnesses—for example high blood pressure illnesses and the like—an assessment of implemented therapy measures can take place in this manner, for example.

The diagnostic evaluation of the sodium values determined with the method according to the invention consequently represents an advantageous possibility for application and an expansion of the physical/technical measurement method itself.

In addition to the method, the present invention also concerns a local coil to acquire sodium image data sets in the method according to the invention. Such a local coil matched to the method according to the invention can in particular be characterized in that, to acquire reference image data of materials with a defined Na+ content, a phantom is integrated into the local coil or the local coil has a particularly exactly shaped receptacle for such a phantom. It is thereby especially advantageous if a placement area is formed by the phantom or is situated directly adjacent to the phantom, as has already been presented.

All statements with regard to the method according to the invention that relate to aspects of the local coil apply analogously to the local coil according to the invention.

The local coil has at least one coil element fashioned to acquire sodium-23 magnetic resonance signals, but the local coil can advantageously and preferably have at least two coil elements fashioned to acquire sodium-23 magnetic resonance signals. A larger number of coil elements are thereby advantageous with regard to the resolution and the like.

As mentioned, it is particularly advantageous for a placement area over the materials of the phantom to be formed by a thin—in particular less than a millimeter thick—layer, in particular a membrane and/or film. The distance between the target region to be acquired and the phantom (in particular the materials delivering the reference data) is then as small as possible, such that the same acquisition conditions prevail. Such an embodiment is in particular useful at high field strengths.

### BRIEF DESCRIPTION OF THE DRAWINGS

FIG. 1 is a flowchart of an exemplary embodiment of the method according to the invention.

FIG. 2 shows an example of possible coil elements of a local coil according to the invention.

FIG. 3 is a perspective view of a local coil according to the invention.

FIG. 4 is a phantom for use in the invention.

FIG. 5A shows the basic design of a two-element local coil that can be used for 23Na MRI of human skin at 7 T.

FIG. 5B shows the local coil of FIG. 5A positioned in a 1H birdcage coil.

FIG. 5C is a flip angle (FA) map to calibrate the spatial sensitivity of the local coil.

FIG. 6 shows (A) proton images for orientation in the anatomy of the lower leg, (B) a 23Na GRE image of the skin, and (C) an image after a normalization.

FIG. 7A is a calibration curve for concentration versus signal intensity.

FIG. 7B shows exponential T1 fits (lines) of free Na+ in water (open squares), of sodium partially bound to 5% of agarose (solid squares), and Na+ in skin tissue (open circles).

FIG. 7C shows an example of mono-exponential T2* decay of Na+ in free aqueous solution, compared with a bi-exponential decay of partially bound Na+ in agarose and skin Na+.

FIG. 8 shows (A) an anatomical reference image (proton image) and 23Na MR images (below) of lower legs of a 25-year old male, and (B) the analogous images measured at a 67-year old male.

FIG. 9 shows graphs regarding the reproducibility (relative to a subject) of the Na+ skin exposures in nine different examination subjects with increasing age (to the right and down).

FIG. 10 shows the skin Na+ content plotted against age, determined via 23Na MRI at 7.0 T.

FIG. 11A shows muscle Na+ content given experimental mineralocorticoid excess.

FIG. 11B shows Na+ concentration in rat muscles without (control) or with DOCA+/−“high salt” (HS).

FIG. 12 shows MR exposures of Na+ content in human tissue, acquired at 3 T.

FIG. 13 shows muscle Na+ content of patients with hyperaldosteronism.

FIG. 14 shows the tissue Na+ content of patients with HTN (refractory hypertension) and in non-hypertensive control groups corresponding to age.

### DESCRIPTION OF THE PREFERRED EMBODIMENTS

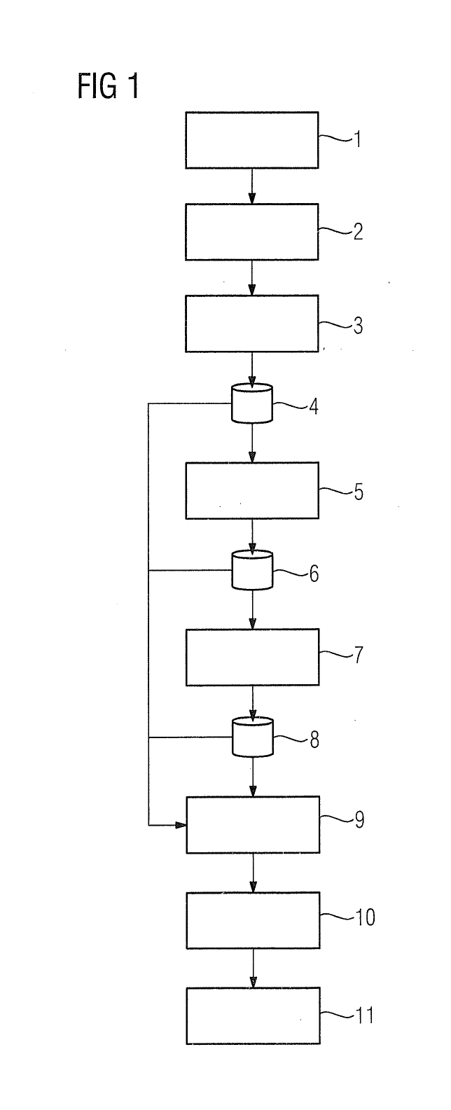

FIG. 1 is a flowchart of a general exemplary embodiment of the method according to the invention. Specific exemplary embodiments are presented in detail in the following with regard to the other figures.

Preparation steps are identified via the boxes 1. Among these are, for example, the registration of the patient with the magnetic resonance device; the removal of unnecessary hardware (a back coil, for example); the deactivation of electrical devices that could possibly affect the measurement; the bearing of the patient (which is preferably conducted “feet first supine”; and the like. The patient should be completely free of metal, and preferably the body region that is ultimately to be measured (a leg, for example) should be free of clothing. After connecting the local coil (in particular a local coil specific to the patient), the patient is suitably positioned so that the desired target region can be acquired by the local coil. For example, if exposures of the lower leg should be made, the largest calf diameter can be positioned in the coil center. If the phantom which should supply the reference data is not already integrated into the local coil, it is now positioned so that it forms at least a portion of the placement area of the target region within the local coil.

In Step 2 a localizer is initially acquired (as is known in principle). For this a suitable sequence of proton imaging can be used.

The acquisition of the sodium image data sets then begins. In the exemplary embodiment shown here, two different types of sodium image data sets are acquired, wherein at this point it is noted that a sodium image data set to be evaluated can also be determined by averaging multiple acquisition processes.

A gradient echo sequence is then initially used in Step 3 in order to acquire at least one first sodium image data set 4 by means of the local coil. For example, a triple use of the gradient echo sequence can take place, whereupon an averaging then takes place in order to determine the sodium image data set 4 that is then evaluated further. It is possible to use multiple echoes in order to suppress portions of bound Na, as noted above.

The acquisition of an additional sodium image data set 6 then takes place in Step 5, in this case using a radial sequence. This has extremely short echo times and consequently generates sodium image data sets 6 that depicts all sodium deposits, independently of whether they are bound or freely mobile.

At this point it is further noted that reference image data that show different materials which have a predetermined Na+ content are also included in the sodium image data sets 4, 6. This is based on the fact that a date set from a phantom, which is preferably integrated into the local coil, is acquired as well. For example, the phantom can include containers, receptacles or the like for NaCl solution, but it is also possible (and, due to the T2* adaptation, preferable) to also use NaCl in 5% agarose as materials (agarose standards).

In Step 7, an anatomy image data set 8 of the target region is then also acquired with the use of a whole-body coil (provided anyway in the magnetic resonance device) or a local coil suitable for proton imaging. It is noted that a water image data set can also be acquired in Step 7 as an additional anatomy image data set via known techniques of fat/water separation (for example a Dixon technique) if a conclusion about the water content should also be made.

In Step 9, regions of interest (ROI) are now determined (preferably automatically), consequently sub-regions of the target region for which the Na+ content should be quantified. There are various approaches for this. For example, one possibility is to initially determine regions of interest in the anatomy image data set 8 (muscles, for example), in particular via known automatic, semi-automatic or manual segmentation methods. Because the anatomy image data set 8 was acquired in the same magnetic resonance device in Step 7 with no change to the position of the patient, a segmentation of anatomy image data set 8 can be transferred to the sodium image data sets 4, 6.

Another variant is to determine the position of a region of interest due to the phantom or other properties visible in the sodium image data sets 4, 6. For example, if the Na+ content in the skin is to be determined, and it is to be assumed that the target region of the patient (the calf, for example) rests directly on the phantom, the skin (which is rich in sodium) can be clearly detected based on the phantom (for example a 0% agarose). For example, the skin can then be located beyond a threshold starting from a rather dark region (in which no sodium is present) and be broadly assumed as an image point, for example when the skin thickness essentially corresponds to the dimensions of an image point. Partial volume effects are thus also kept within limits.

Within the scope of the segmentation in Step 9, known, strongly sodium-containing structures can advantageously also be excluded from further consideration, for example intergrown blood vessels and the like.

At this point it is noted that—when observations of the water level should also be made—the regions of interest can naturally also be transferred to a corresponding water image data set. The background noise (0 kilos water per liter) and, for example, an aqueous sodium solution that is used in the phantom—which then corresponds to one kilo water per liter, for instance—can be used as a calibration standard to determine water values describing a water content.

It is preferred—particularly at high basic field strengths of the magnetic resonance device—to make a B1 correction of the inhomogeneities before the additional evaluation of the sodium image data sets 4, 6. For this purpose, a correction image data set is either already present for every used magnetic resonance sequence, or it is acquired after removal of the patient from the magnetic resonance device. A correction image data set is acquired using the same magnetic resonance sequence (thus in the example the gradient echo sequence and the radial sequence), for example by a suitable subject that has a homogenous Na+ content being positioned as a calibration phantom at the position of the target region. For example, an agarose block with suitable NaCl contents can be used here.

In an optional correction step (not shown in FIG. 1), the sodium image data sets 4, 6 are then divided per image point by the respective associated correction image data set in order to implement the correction of the spatial inhomogeneities with the use of the correction image data set acting as a B1 map.

The calculation of the sodium values then takes place in Step 10. In the simplest case, this can take place via averaging of the image data in the regions of interest, whereupon a linear trend analysis takes place with regard to reference image data of the materials that can be associated with the different Na+ contents. An averaging can naturally also take place for the reference image data, wherein the obtained averaged image value is associated with the Na+ content. If an image value of a region of interest lies between two image values, a linear correlation is assumed and a Na+ content is accordingly determined (for example in the form of a Na+ concentration) for the image value.

In more complicated embodiments, however, it is also possible to determine sodium values for bound or freely mobile Na+ in that a sodium image data set 4 is acquired using multiple echoes so that the content of the bound sodium is suppressed, while the sodium image data set 6 shows the entirety of the sodium. A sodium value for bound Na+ can now also be determined by suitable difference calculation.

It should be emphasized again that the determined sodium values are method-specific contents that are affected by different effects already presented in the general description at the outset. Naturally, it is conceivable to also conduct a calibration in this regard and a conversion of the sodium values into actual Na+ contents (in particular concentrations) in Step 10.

The calculation of the sodium values according to the invention also preferably takes place automatically, but it should be noted that it is also possible (because the reference image data are themselves included in the image) to roughly read out a Na+ content in the shown sodium image data set and the like. The further evaluation then takes place in Step 11, for example in that initially the sodium values are displayed in order to then be diagnostically interpreted further by a physician. It is also possible to implement a computer aided diagnosis (CAD) in order to obtain diagnostic or other information.

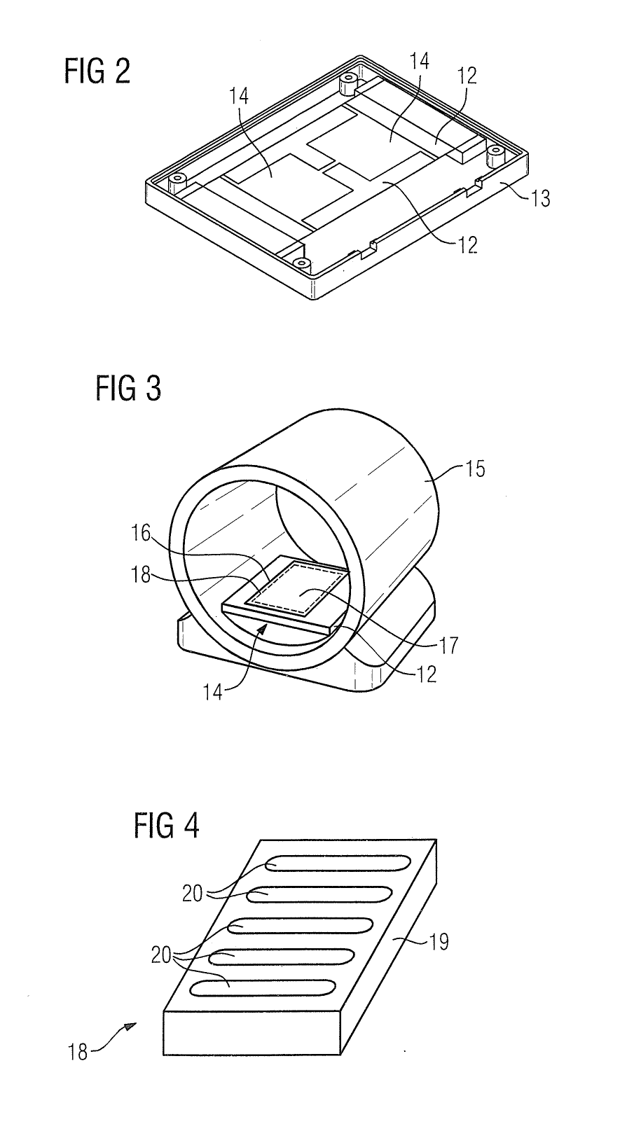

FIG. 2 shows a skeletal structure of a local coil usable within the scope of the present invention. Conductor traces 13, which define the coil elements 14 that are matched to the sodium-23 imaging, run in a suitable coil housing 12 (that here is only partially shown). Two coil elements 14 are presently but, more coil elements 14 can naturally be provided (or, less preferably, only one coil element 14). The coil elements 14 are decoupled by capacitors (not shown).

FIG. 3 shows the local coil 14 according to the invention arranged in a birdcage coil 15 matched to proton imaging. In its upper part, the coil housing 12 clearly has a receptacle 16 that is covered by a thin layer 17 (for example a membrane or film) and in which a phantom 18 (only indicated here) is integrated. The thin layer 17 enables that the target region placed on the formed placement area is placed as close as possible to the phantom 18 and the coil elements 14.

The placement area (like the phantom 18) alternatively may be lower to one side, preferably in the region of a material that should indicate reference image data for no sodium content. On this side is the target region—in particular the skin to be examined as a region of interest—is then located closer to the coil elements 14, which improves the signal-to-noise ratio and additionally ensures that the same imaging conditions are provided for the other materials as for corresponding regions of the target region.

FIG. 4 shows an exemplary embodiment of a phantom 18. This embodiment has a base body 19 in which multiple receptacles 20 are provided as recesses into which the materials (for example thus NaCl solutions or agaroses) can be introduced in order to then be covered by the layer 17. For example, equidistant materials with regard to the Na+ content can be introduced into the receptacles 20, here for example materials with 0, 10, 20, 30 and 40 mM NaCl given five receptacles.

A few specific exemplary embodiments and scientific results that were achieved with the method according to the invention are now presented in detail in the following.

The water content of biological tissue can be measured with proton MRT (¹H⁺). If the transversal relaxation processes are taken into account and if additional local field inhomogeneities are corrected, the signal intensity of proton density-weighted exposures directly maps the water content of the tissue. Alternatively, the water content can also be calculated from the 1H+ peak of proton spectra. The combination of both spectroscopic methods enables the non-invasive determination of the Na+ content and water content in different tissues. Shifts of the internal Na+ distribution are thereby measureable. The practical implementation capability of the described method is subsequently demonstrated in an animal model (see FIG. 11).

FIG. 11 shows the measurement of the Na+ content in the skeletal musculature in rats treated with deoxycorticosterone (DOCA) with the use of dry ashing (right quadricep muscle) or with 23Na+ MR spectroscopy (left quadricep muscle).

An endocrine-dependent hypertonia that simulates Conn syndrome as a form of hypertonia illness in people was induced by the DOCA treatment. The DOCA treatment led to distinct arterial hypertonia that accompanied increase of the body's Na+ content and a Na+ redistribution disruption. Within the scope of this redistribution disruption, an increase of the Na+ content in the skeletal musculature typically occurs. This increase of the muscular Na+ content could be detected non-invasively, ex vivo, with 23Na+ MR spectroscopy.

The left graph (A) in FIG. 11 shows: 23Na+ determination with MR spectroscopy. A phantom tubule with 150 mM NaCl and 5 mM shift reagent was placed in the center of a muscle sample. The shifted phantom sodium signal (left peak) was used as a reference to calculate the Na+ concentration in the muscle (right peak).

The right graphs (B) in FIG. 11 show: Na+ determination in the quadriceps muscle of the rat by means of MR spectroscopy (left) and by means of flame photometry (right). The rats were untreated (control) or DOCA-treated (DOCA) and received tap water (white bars) or 1% salt water (grey bars) to drink.

Sub-millimeter resolution for 23Na MRI in the human skin at 7.0 T

Sodium (Na+) measurement in living bodies is not easy. Techniques of 23Na magnetic resonance imaging (MRI) were used in order to estimate the Na+ content of tissues with high precision.

The suitability of 7.0 T MRI technology was tested on normotensive patients, with concentration on the skin. Transverse slices of the calf were acquired with 23Na MRI using a two-channel monoresonant surface coil array and an optimized gradient echo sequence (GRE) with a sub-millimeter resolution (0.9×0.9) mm2 in the plane within 10 minutes. The skin Na+ content was determined by means of a linear trend analysis of the signal intensities relative to agarose standards with various Na+ concentrations.

The 23Na coil showed a high sensitivity within a distance of approximately 1 cm from the surface. Spatial inhomogeneities were corrected via normalization. To estimate the Na+ content, MRI-specific saturation effects (T1 contributions) and T2* effects were reduced by using agarose in calibration phantoms and long repetition times TR. The acquired 23Na images showed a high contrast for Na+ in the skin compared to lower Na+ values in subcutaneous fat and to a Na+-free environment.

The fluctuations between various examination subjects lay below 6%. Significant, age-dependent differences in the skin Na+ content were obtained between the examination subjects (R2=0.95). It is concluded that human skin Na+ content can be quantified by 23Na MRI at 7.0 T.

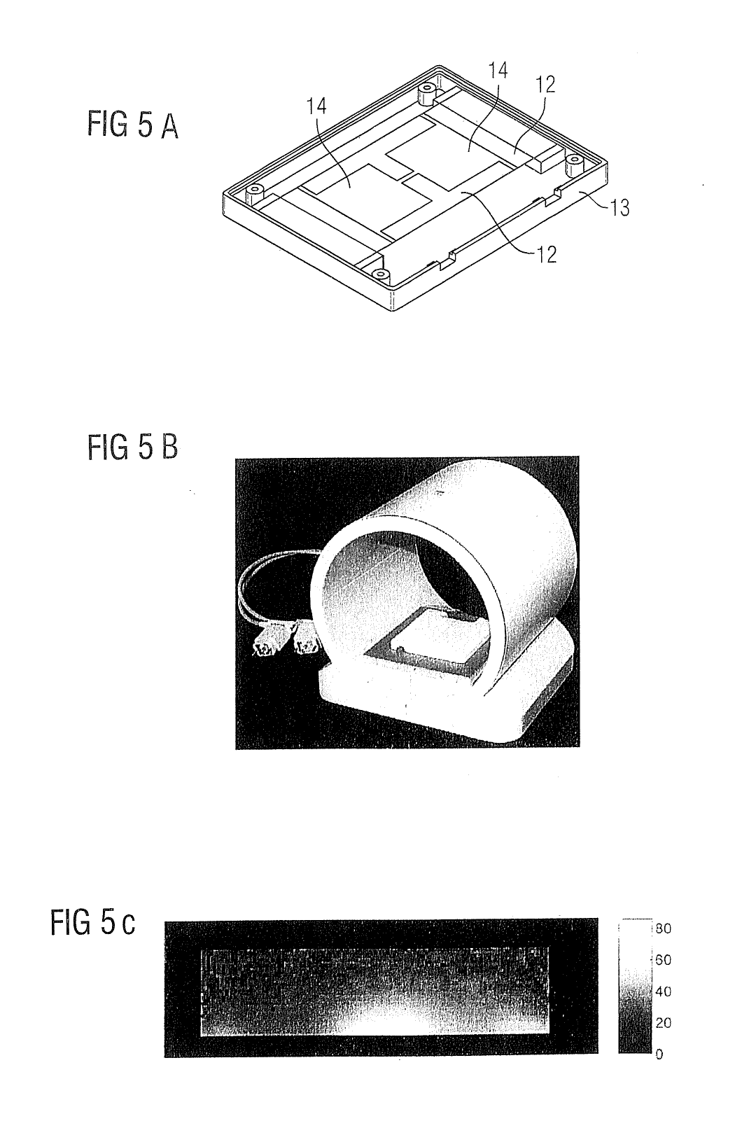

A 23Na radio-frequency coil optimized for an imaging of the human skin has been developed. The coil comprises two loop elements, respectively of a size of (5×6) cm², for instance (see FIG. 5A).

A small coil size and a low resonance frequency (here approximately 78.5 MHz) allow a radio-frequency shielding to be omitted.

The coil elements were decoupled by capacitors. The characteristics of the inventive TX/RX array were examined by means of simulations (electromagnetic field, EMF) and specific absorption rate (SAR)). In volunteer studies (FIG. 5B), the 23Na coil was positioned inside a 1H birdcage coil

Acquisitions of anatomical reference images were made with the coil. For Na+ calibration, an array of 5% agarose gels comprising 0, 20, 40, and 60 mmol/L NaCl was used as an external standard (phantom). The standards were placed on top of the 23Na surface coil. The 20, 40, and 60 mmol/L NaCl standards had dimensions of (10×20×75) mm³. Na+-free agarose was chosen to be thinner (approximately (5×20×75) mm³) in order to be able to position the skin closer to the surface coil, and therefore to achieve a better signal-to-noise ratio (SNR).

Patients were positioned feet first and supine in the MR system. For MRI, the posterior region of the lower leg was positioned onto the external standards. The external standards and the calf were aligned parallel to the z-axis of the MR system to in order to be able to acquire straight, transversal slices free from partial volume effects with tissue components other than skin.

The Na+ signal intensities were evaluated for skin regions directly above the NaCl-free agarose standard because of the higher contrast between skin and the external standard and the sensitivity of the radio-frequency coil that was used in that region.

A standard proton localizer was used (2D FLASH, echo time (TE)=3.7 milliseconds (ms), repetition time (TR)=10 ms, flip angle (FA)=20°, bandwidth=320 Hz/pixel, field of view (FOV)=(192×192) mm², voxel size (0.375×0.375×5) mm³) with what is known as an “average” and a resulting total measurement time (TA) of 2.4 s. 23Na MRI was performed using a GRE sequence that was optimized for sodium imaging with short echo times TE (highly asymmetric echo, 400 μs excitation pulse, TE=2.27 ms, TR=135 ms, FA=90°, bandwidth=280 Hz/pixel, FOV=(128×128) mm², voxel size (0.9×0.9×30) mm³ with 32 “averages” and a resulting total measurement time TA of approximately 10 minutes).

For quantification of the Na+ content, T1 saturation effects were examined. For this purpose, one volunteer was measured several times together with an agarose standard and an aqueous NaCl standard (each 40 mmol/L) using a GRE imaging technique (FA=90°, bandwidth=310 Hz/pixel, FOV=(128×64) mm², voxel size=(1×1×30) mm³, number of “averages”=32, TE=3.47 ms) in conjunction with repetition times in a range from TR=10 ms to TR=200 ms. Furthermore, the bi-exponential T2* decay rates of Na+ in tissue were measured using short echo time techniques, for example as they are described by Lifton R P, Gharavi A G, Geller D S in “Molecular mechanisms of human hypertension”, Cell. 2001; 104:545-556, or by Heer M, Baisch F, Kropp J, Gerzer R, Drummer C in “High dietary sodium chloride consumption may not induce body fluid retention in humans”, Am J Physiol Renal Physiol. 2000; 278:F585-595. Skin, agarose and the aqueous standards were thereby acquired by means of a fast 3D spiral technique (TR=200 ms, FA=90°, bandwidth=200 Hz/pixel, FOV=(128×128) mm², voxel size=(1×1×1) mm³, number of “averages”=2 with echo times ranging from 0.05 ms to 20 ms. Thirty transversal slices were averaged in order to obtain a sufficient SNR.

Flip angle maps (FA maps) were generated in order to measure the transmit sensitivity profile (B1+) of the 23Na coil. For this purpose a cuboid phantom comprising 40 mmol/L NaCl, with dimensions of 30 mm×105 mm in-plane and 75 mm thickness, was arranged at the same location as the agarose standard in the volunteer measurements. A double angle method was implemented using the same sequence parameters as in the patient studies, but with 200 “averages”, TR=200 ms and FA1,2=45°/90°, and therefore with a total measurement time of 2.8 h.

Surface coil B1-inhomogeneities in the in vivo images were corrected by means of B1-maps which were derived from the cuboid agarose phantom. For this purpose, the uncorrected images of the human skin were divided by the B1 map. This B1-correction is justified by the low resonance frequency of the sodium, the use of a transmit/receive coil in what is known as “quadrature mode 10” and the homogeneity of the large agarose phantom.

The signal intensities which were derived from the NaCl-free agarose standard formed the basis for the determination of background noise level. The tenfold value of that level was set as a limit value that typically encompasses all pixels which contain skin. The region of this region of interest (ROI), which was arranged above the 0 mmol/L agarose standard comprising NaCl, was evaluated as skin Na+ content. The mean signal intensity of the skin was compared with the intensity values of agarose standards comprising 20, 40, and 60 mmol/L NaCl in a linear trend analysis.

The standard deviation of the signal intensities in the ROI in the skin was used in order to define the standard deviation of the Na+ content in the skin. The reproducibility of the measurements was determined by repositioning of the calf in five successive, independent measurements of the same volunteer. The “intra-subject”standard deviation—thus the standard deviation with regard to the same examination subject—was defined as the variance of five successive measurements of one volunteer.

With the coil described above, the best signal-to-noise ratios were achieved at a transmit voltage of 25 V. At this transmit voltage a nominal flip angle FA of 90° was achieved in the central region of the coil. An FA map was created using phantom measurements (FIG. 5C). The FA map shows the high sensitivity of the coil in its central region and in regions close to the surface coil elements. The flip angle decays up to approximately 50% per 1 cm distance from the surface.

### Results

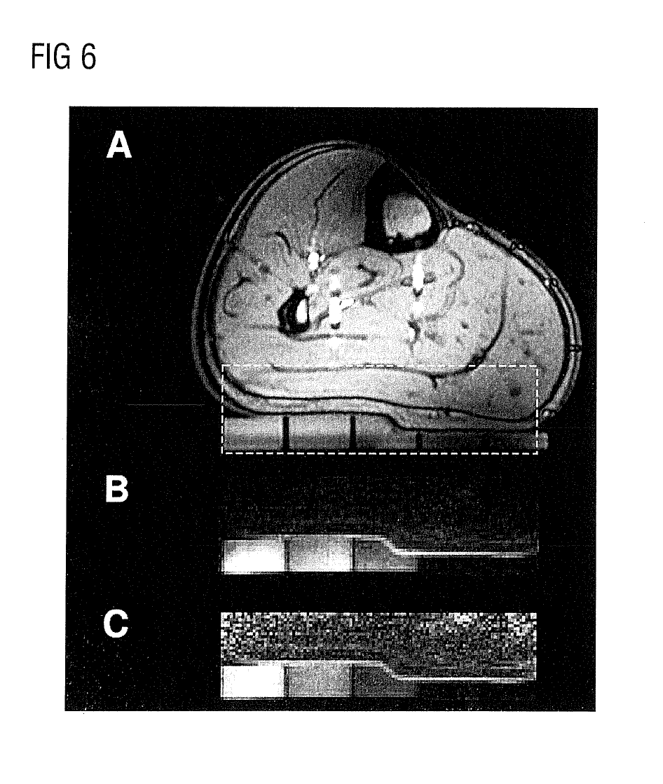

A proton image was used for orientation in the anatomy and to optimize the positioning of the skin and the FOV for sodium imaging (FIG. 6A). A raw image (FIG. 6B) acquire with the 23Na coil provided a spatial sub-millimeter resolution in-plane. The signal intensities of the 20, 40, and 60 mmol/L Na+ standards could be very well distinguished from each other. The Na+ signal in the thin layer of skin shows a high contrast versus the 0 mmol/L NaCl agarose. Also, the skin layer was very well delineated from the subcutaneous fat layer. A normalization of the Na+-image by means of the flip angle map (FA map) reduced B1 inhomogeneities (FIG. 6C). Intensity values of the external standards (which are proportional to Na+ content) could therefore be compared with the average signal intensity of the skin by means of a linear trend analysis.

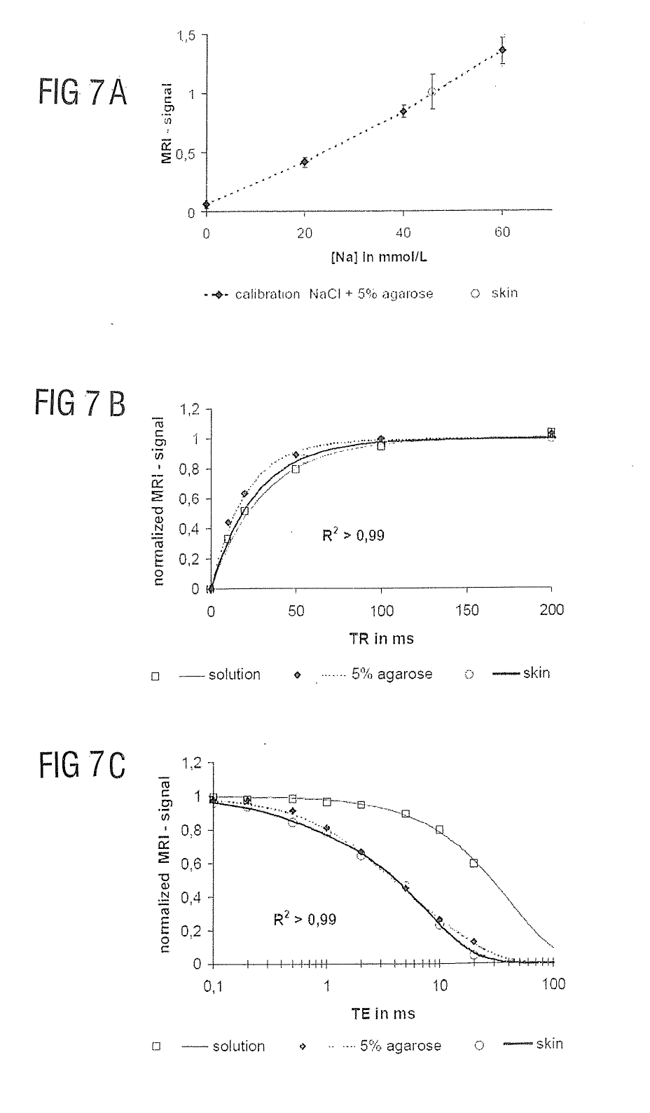

A Na+ content between approximately 40 mmol/L and approximately 60 mmol/L could be established in healthy volunteers. This range of Na+ content fell into the linear region of the calibration curve (FIG. 7A). T1 saturation effects in human skin (T1=27±2 ms) were comparable to that of the aqueous 50 mmol/L NaCl (T1=31±3 ms) standard and the 50 mmol/L NaCl in 5% agarose (T1=20±2 ms) standard. Therefore, agarose was used as an external standard in order to be able to use shorter repetition times without compromising the spin density weighting which is necessary for Na calibration (FIG. 7B). At repetition times >100 ms, the error in the concentration calibration of the skin using agarose standards was well below 5%. More challenging is the reduction (or even elimination) of the T2* contributions to the signal intensity, as is shown in FIG. 7C. Free aqueous solution decays mono-exponentially with a relatively long time constant of T2*=41±4 ms. In contrast to this, partially bound Na+ is characterized by a bi-exponential decay which includes a fast and a slow component (agarose: T2^{*}_{fast}=2.3±0.5 ms, T2*_{slow}=13±2 ms, skin: T2*_{fast}=0.5±0.3 ms, T2*_{slow}=7.6±0.5 ms). In order to reduce the contributions to the calibration that are caused by T2* effects, external standards which imitate the T2* relaxation properties of the tissue were used. Agarose satisfied this condition and was therefore chosen as an external standard.

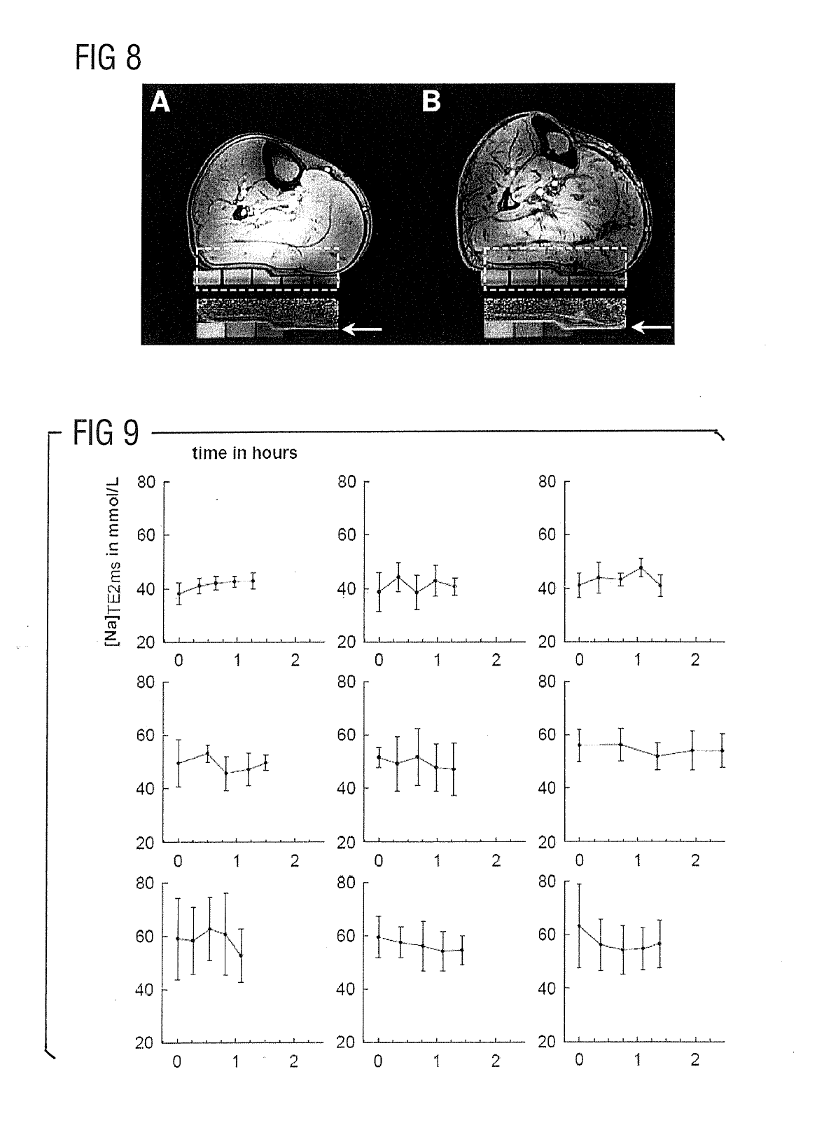

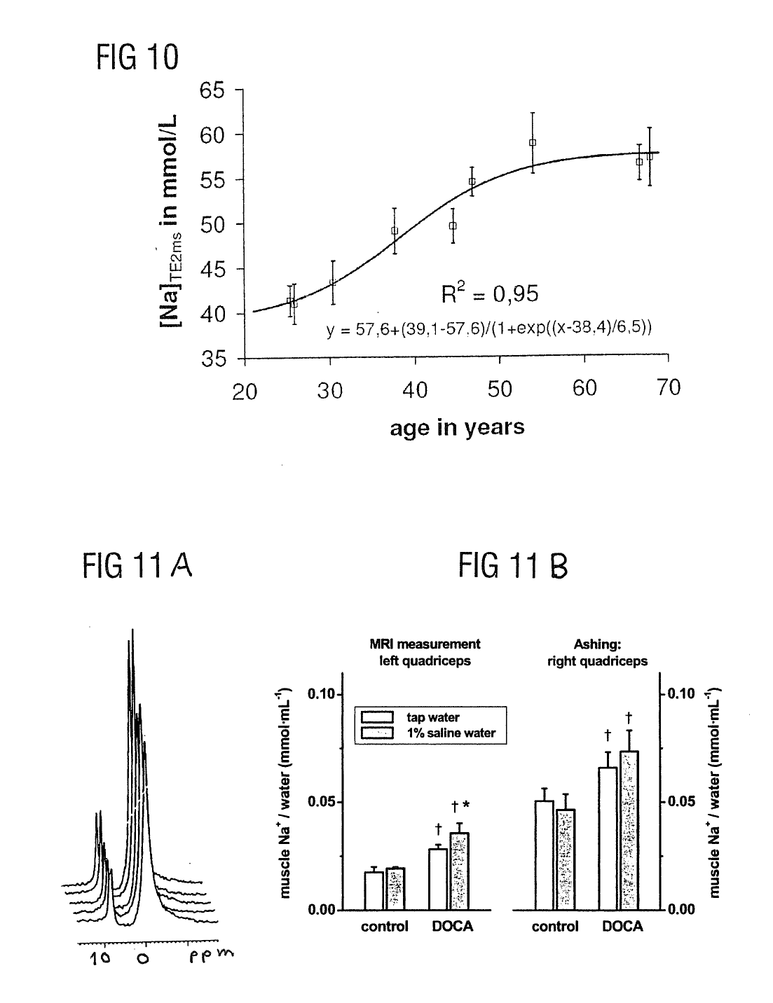

In the in vivo studies, differences in the Na+ content of the skin were established between the examination subjects (“inter-subject”). The Na+ content of a 25 year-old male was 41±2 mmol/L (FIG. 8, image A, bottom). In comparison to this, the skin Na+ content of a 67 year-old male (FIG. 8, image B, bottom) was approximately 1.4-times higher (57±3 mmol/L). In all examination subjects, the reproducibility of the results of the Na+ content of the skin was determined for the respective examination subject (“intra-subject”). The fluctuations for all examination subjects were hereby respectively below 6% (FIG. 9). The previous 23Na MRI in vivo measurement data, which include nine examination subjects ranging in age from 25 to 68 years, suggest an age-dependent increase of the skin Na+ content (FIG. 10). The correlation can be well depicted by means of a sigmoidal Boltzmann fit with a maximum slope at 38±5 years and a regression coefficient of R2=0.95.

Compartmentalized Na+ stores can thus be measured with high precision by means of 23Na MRI, even in living examination subjects. The use of 23Na MRI in connection with 7.0 T MR systems yields advantages in the sensitivity and spatial resolution in comparison to 3.0 T MR systems. With 23Na MRI at 7.0 T, the higher sensitivity of the surface coil enabled acquisition of 23Na MR images at an in-plane resolution of less than 1 mm in the thin layers of the skin. The enormous Na+ content of the human skin could be shown for the first time via this improved resolution. In the sensitivity range of the coil, the skin tissue showed a high signal in a thin layer of approximately 1 mm between the agarose standards and the nearly Na+-free subcutaneous fat tissue.

The Na coil and its resolution can be further improved in that, for example, the size of the loop elements is reduced and/or the number of loop elements is increased. The selected slice thickness of the measurements can be even further reduced in order to increase the resolution. The inventive method thus would also be more robust for clinical use and diagnoses of salt-sensitive hypertension.

The use of larger slice thicknesses for the acquisition of transverse slices of the skin in the lower leg region requires an optimized positioning of the skin and agarose parallel to the z-axis of the magnet of the MR system in order to reduce partial volume effects, and therefore a mixing of the Na-signals of the skin and the Na+-free environment and low-Na+ subcutaneous fat tissue.

Despite a lack of fast and appropriate B1-mapping techniques that are suitable for human studies, the presented normalization method works sufficiently well for a concentration calibration. A further reduction of the repetition times (and therefore of the total measurement time) is not advisable if spin density contrast is necessary.

In the conducted studies, the repetition time TR was already limited by SAR requirements. The T2* contrast problem that was addressed above was minimized by the use of agarose standards that exhibit a T2* relaxation decay which lies within the range of the T2* decay of skin tissue.

The Cartesian GRE sequence that was used was optimized to the geometry and conditions of the in vivo measurements. It is likewise conceivable to use fast 2D or 3D projection imaging techniques in order to be able to measure the signal components of rapidly decaying 23Na and to further reduce the cited T2* effects.

With the method according to the invention it is possible to analyze the previously unknown mechanism of Na+ balancing even in humans

FIG. 5 shows: (A) the basic design and layout of the two-element transmit/receive surface coil which can be used for 23Na MRI of the human skin at 7 T. (B) 23Na surface coil positioned in a 1H birdcage coil, the latter of which can be used for acquisition of anatomical reference images. For calibration of the concentrations, agarose phantoms (as standards) with concentrations of 20, 40 and 60 mmol/L NaCl were mounted on the 23Na coil. (C) a flip angle (FA) map as it was determined from a 40 mmol/L NaCl agarose phantom for calibration of the spatial sensitivity of the coil. The FA map shows a high sensitivity of the coil near the surface. The flip angle (FA) decays to approximately 50% at a distance of approximately 1 cm from the surface.

FIG. 6 shows: (A) proton images serve for the orientation in the anatomy of the lower leg lying on an array of agarose gel standards with different NaCl concentrations of 0, 20, 40, and 60 mmol/L (from the right to the left). The dashed line surrounds the FOV of the 23Na image. The 23 Na surface coil was positioned below the agarose standards. (B) 23Na GRE image of skin. The bright white line represents the high Na concentration in the thin skin layer. (C) After a normalization, the standards can be used in order to calibrate the Na+ content of the tissue.

FIG. 7 shows: (A) the concentration-to-signal intensity calibration curve is linear at concentrations >20 mmol/L. The Na+-content of the skin was determined by means of a linear trend analysis of tissue greyscale values. The black squares represent the Na+-content in agarose; open circles represent examples of measurements of the skin. (B) Exponential T1-fits (lines) of free Na+ in water (open squares), of sodium partially bound to 5% of agarose (solid squares), and Na+ in skin tissue (open circles). For repetition times TR>100 ms, the error in signal calibration is less than 5%. (C) A representative mono-exponential T2*-decay of Na+ in free aqueous solution compared to a bi-exponential decay of partially bound Na+ in agarose and skin Na+. The measurement data were acquired by means of an ultra-short TE imaging technique using the surface coil shown above. In skin Na+ measurements, the Na+ agarose calibration at echo times >0.1 ms was superior to the aqueous Na+ standards.

FIG. 8 shows: (A) an anatomical reference (proton image) and 23Na MR images (below) of lower legs of a 25 year-old male and (B) the analogous images measured at a 67 year-old male.

The upper panels show the anatomical structures which were determined by means of 1H MRI. The lower panels show the density-corrected 23Na MR images of the same skin and the agarose gel standards with increasing Na+ content.

FIG. 9 shows: “intra-subject” reproducibility of Na+ skin acquisitions in nine different examination subjects with increasing age (to the right and downward).

FIG. 10 shows: the skin Na+ content plotted against age, determined via 23Na MRI at 7.0 T. The preliminary results indicate a sigmoidal correlation between Na+ content of the skin and age (R2=0.95).

### 23Na MRI to Examination Functional Disruptions of the Internal Na+ Balance

Disruptions of the physical volume in edematous states and high blood pressure are coupled with a disrupted Na+ regulation in the body. Precise measurements of Na+ in tissue are possible by means of ashing and atomic absorption spectrometry and have provided unexpected results that shed new light on Na+ balance in the entire body and Na+ storage in tissue. However, these methods cannot be used in everyday clinical environments.

By means of 23Na MRS (magnetic resonance spectroscopy) and MRI (magnetic resonance imaging) at 3 Tesla (T), 7 T, and 9.4 T, it was sought to quantify Na+ content in skin and skeletal muscle. 23Na MR data were compared with an actual tissue Na+ content in animal and human tissue which was determined by means of chemical analyses. The tissue Na+ content in patients with aldosteronism and in patients with high blood pressure (refractory hypertension, HTN) was then quantified non-invasively in comparison with control tissues.

Skin and muscle Na+ content (determined via 23Na MRI) showed a high precision within the method and appeared similar to Na+ measurements by means of chemical analysis.

An increase of 29% in muscle Na+ content could be established in patients with high blood pressure. This excess if Na+ in muscle could be successfully reduced without accompanying weight loss. Male HTN patients showed increased muscle Na+ content. Spironolactone treatment reduced the Na+ content back to control levels. Female HTN patients had increased Na+ content in their skin.

By means if 23Na MRI it is possible to quantify hidden Na+ stores in humans or animals which otherwise remain undetected. 23Na+ MRI can be used in order to examine the correlations between Na+ accumulation, Na+ distribution, hypertension, and edema.

### 23Na MRI Quantification of Tissue Na+ Content in Animals

Twenty rats were randomly assigned to four groups and received either tap water (control group) or 1% saline (“high salt”) to drink. Ten rats additionally received a treatment with deoxycorticosterone acetate (DOCA) for four weeks. Directly after terminating the rats, both quadriceps muscles of each animal were removed. The Na+ concentrations of the left quadriceps were determined by means of chemical analysis, while the Na+ content of the right quadriceps was analyzed by means of 23Na MR spectroscopy at a 9.4 T MR installation with a micro-imaging gradient system. The 23Na spectra were determined as 128 time-averaged free induction decays (FID) with a repetition time of 1.5 s. The total Na+ content of the muscle was calculated by integrating the area under the associated signal and was compared with the area integrated over a shifted Na+ reference signal.

### 23Na MRI Quantification of the Tissue Na+ Content of Humans

Amputated upper and lower human legs were cut into slices with a thickness of >3 cm and were deep-frozen. For ex vivo 23Na MRI measurements the slices were thawed and heated to approximately 20° C. Directly after 23Na MRI measurements, the examined regions (ROIs) were dissected, underwent an ashing procedure and subjected to chemical analysis.

The tissue Na+ content was examined non-invasively by means of a 3.0 T clinical MR system. 23Na MRI was performed with a GRE sequence (2D-FLASH, total measurement time=13.7 min, TE=2.7 ms (amputated legs)/TE=2.07 ms (in vivo lower legs), TR=100 ms, flip angle=90°, 128 averages, resolution 3×3×30 mm³) and a frequency-adapted, monoresonant TX/RX birdcage knee coil. 1H imaging was performed with the body coil of the MR system using a scout sequence of the system (123.2 MHz, 2D-FLASH, total measurement time=4 s, TE=4 ms, TR=8.6 ms, flip angle=20°, 2 averages, resolution 0.375×0.375×7 mm³). For the ex vivo analysis, the slices of the amputated lower legs were fixed by means of a polystyrene holder which comprised 50 ml tubes with 10, 20, 30, 40 and 50 mM NaCl as calibration solutions. For in vivo measurements, the examination subjects positioned their lower legs in the center of a 23Na knee coil. 23Na MRI greyscale measurements of standard solutions with increasing NaCl concentration (10, 20, 30 and 40 mM) served to calibrate the relative tissue Na+ content.

Measurements at a 7 T MR system were conducted with a 23Na two-channel surface coil (as it is described above, for example) which was arranged in the 1H basic coil of the system for the anatomical imaging.

23Na MRI was performed with a GRE sequence (2D-FLASH, imaging frequency=78.6 MHz, total measurement time=10 min, TE=3.47 ms, TR=150 ms, flip angle=90°, 64 averages, resolution 1×1×30 mm³). 1H imaging was conducted by means of the scout sequence (imaging frequency=297.1 MHz, 2D-FLASH, total measurement time=8 s, TE=4 ms, TR=8.6 ms, flip angle=20°, 4 averages, resolution 0.375×0.375×7 mm³). The lower legs were arranged over 4% agarose standards with 0, 20, 40 and 60 mM NaCl for quantification of the skin Na+ content.

### Results

Results of measurements of muscle Na+/water concentrations of either 23Na MR spectroscopy or chemical analysis after ashing with atomic absorption spectrometry (AAS) in control rats or in rats with DOCA treatment (which received either tap water or 1% saline to drink (FIG. 11)) were compared. DOCA treatment led to increased Na+/water concentration in the muscles. The 23Na MR values were somewhat lower, but the effect of the mineralocorticoid excess can be detected and the muscles with excess Na+ accumulation could be correctly identified.

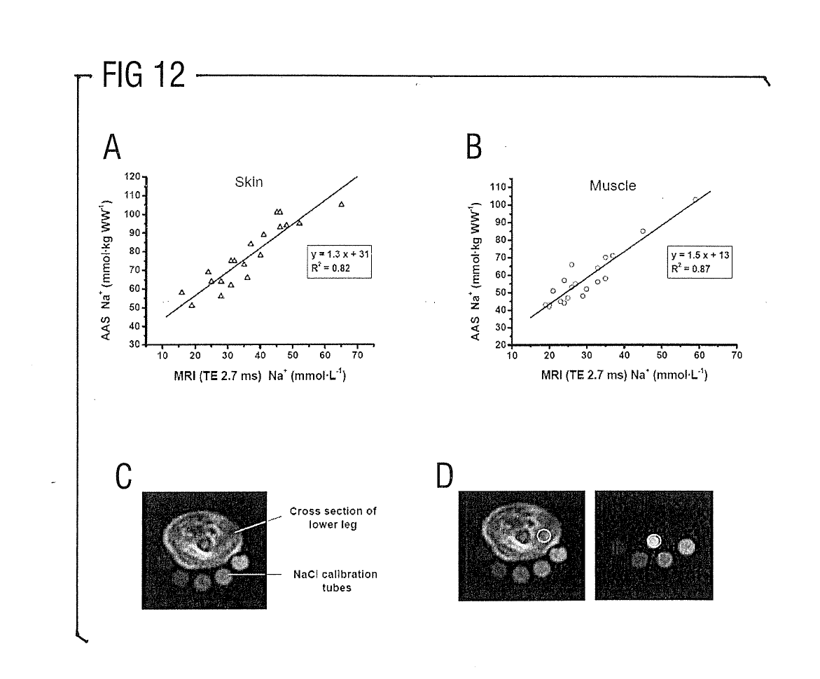

Human tissue from patients who required amputation of the lower extremities was examined next. By means of the 23Na MR imaging method, the Na+ content was initially measured non-invasively and quantified in that signal intensities to tubes with increasing Na+ content were referenced (FIG. 12). The tissue was then chemically analyzed in order to relate the non-invasive determination by means of 23Na MRI to the “gold standard” technique for electrolyte quantification. Similar to animal tissue, the chemical analysis showed that Na+ content in human tissue was significantly more variable than expected (skin Na+ content: 77±16 mmol/kg; muscle Na+ content: 57±15 mmol/kg; n=21) and showed a broad distribution, while plasma Na+ concentrations in the same patients were stable within a very narrow distribution (138±4 mmol/L).

Again, the 23Na MRI measurements resulted in lower values than the chemical analysis; however, a close correlation existed between the two methods (FIG. 12). Moreover, repeated 23Na MRI measurements of the same cross section showed a high precision within the method, with a standard deviation of only 1.4% for both skin measurements and muscle measurements. 23Na MRI is thus a reliable method for non-invasive determination and monitoring of the Na+ distribution in humans as well.

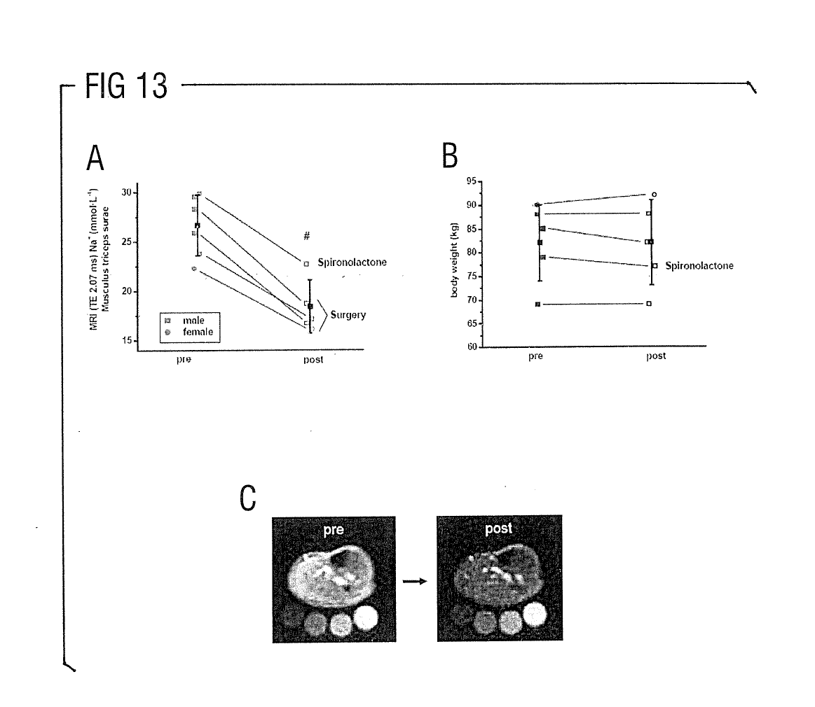

Patients with aldosteronism who had not yet begun spironolactone treatment or not yet had surgery on an adrenal adenoma (FIG. 13 and following table) were recruited next.

Groups

| | male, | female, | male, | female, |
|---|---|---|---|---|
| Parameter | normotensive | normotensive | hypertensive | hypertensive |
| Number n | 17 | 13 | 23 | 11 |
| Age y | 62 ± 7 | 60 ± 7 | 65 ± 8 | 63 ± 7 |
| Weight kg | 77.1 ± 9.9 | 66.2 ± 7.5 | 89.0 ± 13.0 | 83.6 ± 17.3 |
| BMI kg/m² | 24.7 ± 3.0 | 23.8 ± 3.0 | 29.1 ± 4.5* | 29.5 ± 5.0* |
| Systolic | 125.5 ± 9.0 | 119.1 ± 8.8 | 143.8 ± 18.5* | 135.3 ± 16.1* |
| pressure mmHg | | | | |
| Diastolic | 80.4 ± 5.9 | 74.8 ± 6.4 | 82.5 ± 10.5 | 78.0 ± 10.1 |
| pressure mmHg | | | | |
| MAP mmHg | 95.8 ± 5.9 | 90.3 ± 4.1 | 104.5 ± 11.6* | 98.9 ± 10.7* |
| Anti-hypertensive | 0 | 0 | 4.2 ± 1.2 | 3.8 ± 0.8 |
| medication n | | | | |
| Aldosterone pg/mL | 31 ± 20 | 41 ± 23 | 91 ± 46* | 63 ± 31 |
| Aldo/renin ratio | — | — | — | — |
| Creatinine mg/dL | 0.96 ± 0.11 | 0.77 ± 0.13 | 1.11 ± 0.14* | 0.82 ± 0.16 |
| Serum Na+ mmol/L | 140 ± 1.6 | 140 ± 1.2 | 140 ± 2.8 | 139 ± 2.1 |
| Serum K+ mmol/L | 3.9 ± 0.2 | 3.8 ± 0.2 | 3.8 ± 0.4 | 3.7 ± 0.4 |
| Na+ spot urine | 141 ± 66 | 157 ± 91 | 94 ± 40* | 126 ± 81 |
| mmol/g creatinine | | | | |
| K+ spot urine | 62 ± 22 | 95 ± 29 | 55 ± 18 | 77 ± 34 |
| mmol/g creatinine | | | | |
| Albumin spot urine | 4 ± 2 | 18 ± 38 | 25 ± 35* | 12 ± 19 |
| mg/g creatinine | | | | |

| | Aldosterism | Aldosterism |
|---|---|---|
| Parameter | pre | post |
| Number n | 5 | 5 |
| Age y | 52 ± 13 | 52 ± 13 |
| Weight kg | 82.2 ± 8.5 | 81.6 ± 9.1 |
| BMI kg/m² | 27.0 ± 4.0 | 26.9 ± 4.5 |
| Systolic | 149 ± 12 | 133 ± 20 |
| pressure mmHg | | |
| Diastolic | 84 ± 10 | 83 ± 8 |
| pressure mmHg | | |
| MAP mmHg | 108 ± 9 | 101 ± 11 |
| Anti-hypertensive | 3.0 ± 1.0 | 3.0 ± 1.9 |
| medication n | | |
| Aldosterone pg/mL | 330 ± 133 | 43 ± 23^{\|§} |
| Aldo/renin ratio | 171 ± 50 | 5 ± 4^{\|§} |
| Creatinine mg/dL | 1.17 ± 0.67 | 1.39 ± 0.87 |
| Serum Na+ mmol/L | 142 ± 2.3 | 139 ± 2.7 |
| Serum K+ mmol/L | 3.0 ± 0.3 | 4.3 ± 0.6^{\|} |
| Na+ spot urine | 85 ± 107 | 99 ± 38 |
| mmol/g creatinine | | |
| K+ spot urine | 47 ± 15 | 59 ± 22 |
| mmol/g creatinine | | |
| Number n | 48 ± 58 | 15 ± 21 |

It was assumed that aldosterone would influence storage of Na+ in muscle similar to as in the rats treated with DOCA. Males with aldosteronism had a 29% higher muscle Na+ content than normotensive persons (27.5±2.6, n=5 vs. 19.6±2.7 mmol/L, n=17). Four of five patients with primary high blood pressure had an adenoma removed and were examined postoperatively. A fifth patient was examined after initiation of spironolactone treatment, since a sampling and imaging of the venous adrenal gland showed no discrete adenoma.

The resulting change in the internal Na+ distribution—which otherwise remains unnoticed, even given examinations of serum Na+ concentrations and fluctuations of the body weight—can be detected simply by means of non-invasive 23Na MRI. Operations and/or spironolactone treatment reduce muscle Na+ content by approximately 30%. On the assumption that the muscle mass corresponds to 43% of total body weight, the normalization of the aldosterone levels after an operation mobilized 400-450 mmol Na+ from the muscle. This negative Na+ balance should have been accompanied by a 2-3 liter water loss if the Na+ were mobilized as osmotically active from extracellular spaces. However, neither a significant change in body weight nor a significant change in the serum Na+ concentration could be established (FIG. 13, tables above). This indicates that aldosteronism [leads] to a water-free Na+ storage in humans, either by means of osmotically inactive Na+ storage, local hypertonicity, or via intracellular osmotically neutral Na+/K+ exchange.

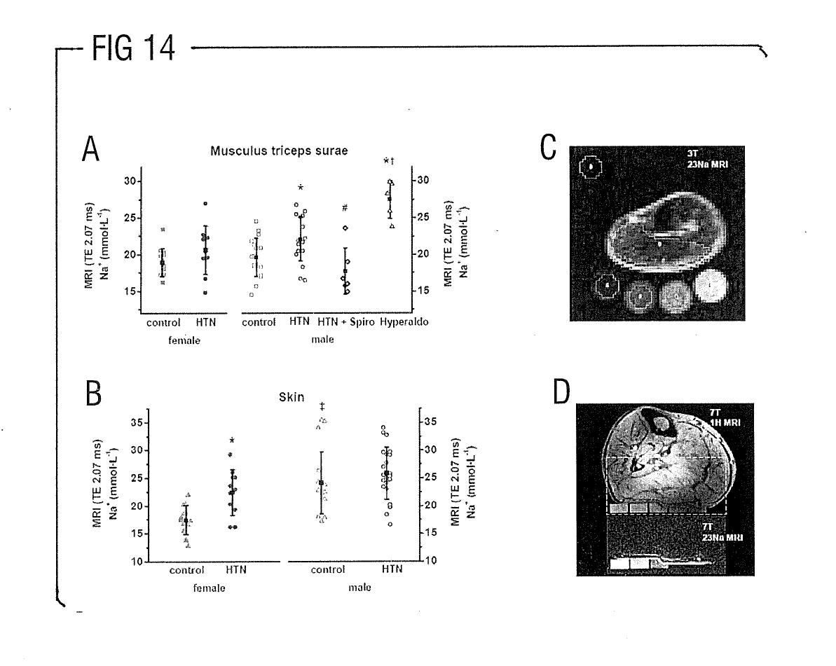

Next, patients were examined, eleven HTN females (female hypertensive) and 23 HTN males (male hypertensive) who exhibited elevated blood pressure even after ingesting three or more different classes of antihypertensive drugs. The Na+ concentrations were likewise examined in 13 normotensive females (female normotensive) and 17 normotensive males (male normotensive) who were approximately the same age as the HTN patients (table above). It turned out (FIG. 14A) that the Na+ content in calf muscle (M. triceps surae) was significantly higher in HTN males in comparison to males of the control group (19.6±2.7, n=17 vs. 22±3.1 mmol/L, n=17), while the serum Na+ values were not different. In contrast to this, a subgroup of HTN patients who were treated with spironolactone showed a decrease in the Na+ content (17.6±3.2, n=6; versus 22±3.1 mmol/L, n=17). Patients with aldosteronism displayed the highest muscle Na+ content of all groups in the study. No difference in the muscle Na+ content could be established in females with HTN.

Since experimental, salt-sensitive hypertension is accompanied by an Na+ storage in the skin, the Na+ content of the skin was also measured with 23Na MRI. A higher skin Na+ content was established in HTN females than in control groups, while in males no difference could be established (FIG. 14B).

Moreover, a higher skin Na+ content was established in normotensive males compared with normotensive females. This gender difference in skin Na+ content was accompanied by higher blood pressure in males compared with females. A quantification of the skin Na+ content by means of 3 T 23Na MRI measurements was hereby limited to a spatial resolution of 3×3×30 mm³. This resolution was not sufficient in order to differentiate between cutaneous and subcutaneous skin Na+ content (FIG. 14C). In contrast to this, measurements at 7 T show a clear delineation of the skin with 1H MRI and 23Na MRI with a markedly higher resolution of 1×1×30 mm³, and allow a differentiated analysis of cutaneous and subcutaneous skin Na+ content (FIG. 14D).

Stores of Na+ in the body can thus be monitored noninvasively by means of 23Na MRI, both in animals and in humans.

It has been shown that considerable quantities of Na+ are stored in muscle without an accompanying fluid retention or changes in the serum Na+ content in patients with aldosteronism, and in patients with high blood pressure refractory hypertension.

23Na MRI is thus a tool in order to be able to examine disruptions in the salt/water balance in a living body, with accumulation sites in the body being detected as well.

23Na MRI in connection with a 3 T MR system was sufficient in order to be able to monitor Na+ accumulation in muscles. A precise quantification of the skin Na+ content was limited by spatial resolution.

23Na MRI in connection with a 7 T MR system showed promising evidence that skin measurements can be improved substantially (see above as well) in order to be able to examine skin Na+ storage.

The non-invasive quantitative determination of disturbances in Na+ metabolism provides new perspectives for patient care and in patient-oriented research.

The possibility of measuring the tissue Na+ content simplifies an estimation of the influence of the environmental factor of salt on cardiovascular diseases. Compared to 24-h urine samples, a direct determination of Na+ accumulation in tissue with 23Na MRI delivers a better controlled and more robust indication, which can clarify potential advantages of diuretic drug treatment or dietary salts. Projected effects of dietary salt restrictions can be measured directly by means of 23Na MRI, and can therefore lead to a more efficient and certain treatment of high blood pressure.

Furthermore, 23Na MRI measurements of muscle Na+ content in patients with hypertension can help identify those patients with underlying aldosteronism.

Muscle Na+ content decreases with a successful adenoma operation or blockade of the mineralocorticoid receptor. Therefore, a 23Na MRI quantification can serve for follow-up examination of patients who were treated for aldosteronism. In the event of recurrence of an aldosterone-producing tumor, a new rise in muscle Na+ content is expected that can be detected with the inventive method.

Furthermore, a phenotyping of the Na+ metabolism by means of 23Na MRI quantification of tissue Na+ storage can help in better understanding and monitoring treatments of Na+ and water retention. The latter is particularly relevant for patients with hepatic, cardiac and renal edema and for dialysis patients.

Briefly, 23Na MRI allows non-invasive identification of hidden Na+ stores in humans and animals which has previously escaped notice. The tool that is provided not only promises not only a better understanding of the relationships between Na+ accumulation, body Na+ distribution, arterial hypertension and edematous disease; it can also help to better identify individual patients who can be especially responsive to reductions of the administration of dietary salt and to diuretic therapy.

23Na MRI measurements can enable a utilitarian, “personalized” therapy of Na+ disorders.

As was already stated above, FIG. 11 shows the muscle Na+ content given experimental mineralocorticoid excess. (B) Na+ concentration in rat muscle without (control) or with DOCA+/−“high salt” (HS) was measured by means of MR spectroscopy or by means of chemical analysis (AAS: atomic absorption spectrometry) after ashing. DOCA+HS increases the muscle Na content; MR spectrometry determines this increase in the same rates (†P(DOCA)<0.05; *P(salt)<0.05). (A) MR spectrograms of five rat muscle samples (right peak). The calibration signal was derived from a 150 mmol/l NaCl standard with what are known as a shift reagent, which results in the shifted calibration signal (left peak) from which the Na+ content of the muscle was calculated.

FIG. 12 shows: MRI measurements of the Na+ content in human tissue, acquired at 3 T. An ex vivo cross sectional analysis of the human lower leg (amputation specimen) by means of 23Na MRI (x-axis) followed by a chemical analysis (y-axis) of the same tissue in skin (A) and muscle (B). The MRI measurements are uniformly lower than the chemical analysis, but the similarity is high. Representative ex vivo 23Na MR images of human lower legs are shown in (C). The cross section is surrounded by calibration tubes with increasing (10-50 mmol/l) Na+ concentration. An increased Na+ content is characterized by an increased intensity of the grey value (D). After 23Na MR examination, the actual Na+ content in the samples was verified chemically. TE: echo time.

FIG. 13 shows: muscle Na+ content of patients with hyperaldosteronism. MRI measurements of the muscle Na+ content of one female and 5 males with primary aldosteronism is shown, with follow-up in 5 patients after the operation (A). The treatment decreased the muscle Na+ content by 30% (26.1±2.6, n=5 vs. 18.4±2.7 mmol/L, n=5). (B) Despite remarkable decrease of the Na+ content, the treatment did not change the body weight. Representative images (C) show a marked reduction in 23Na MR signal intensity which was measured after removal of the aldosterone-secreting tumor (#P(treatment)<0.05). TE: echo time.

FIG. 14 shows: tissue Na+ content in patients with refractory hypertension (HTN) and in age-matched non-hypertensive control groups. Muscle Na+ content in females and males with HTN. males—but not females—with HTN show an increased muscle Na+ content compared to the control group (P<0.05). Spironolactone reduced the muscle Na+ content (A). The muscle Na+ content was lower in the control group and in HTN patients compared to patients with aldosteronism. The skin Na+ content was higher in HTN females in comparison to the control group, while no differences were shown in males (B). Males had higher values than females. Na+ content in skin and muscle was determined via the signal intensity of the tissue in comparison to the control tubes filled with saline. The resolution of approximately 3×3×30 mm³ does not allow a differential analysis of cutaneous and subcutaneous skin Na+ content by means of 23Na MRI at 3 T (C). Anatomical structures (including skin thickness) were determined by 1H MRI at 7 T (D). Below the 1H image is the 23Na MR image of the same skin at 7 T, compared to the agarose gel standards with increased Na+ content (rectangles). The improved resolution of approximately 1×1×30 mm³ at 7 T allows a better assessment of skin Na+ content in humans. TE: echo time. *P(HTN)<0.05; #P(spiro)<0.05 vs. HTN; †P(hyperaldo)<0.05 versus HTN, ‡P(gender)<0.05.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to determine at least one sodium value describing 23N+ content in at least one region of interest in a target region in the body of a patient, comprising:
operating a magnetic resonance data acquisition unit with a sequence configured for sodium-23 imaging sequence to acquire at least one sodium image data set of a target region of the body of a patient in the magnetic resonance data acquisition unit, said at least one sodium image data set comprising image data dependent on a presence of sodium in said target region;
in a computerized processor, defining at least one region of interest for which a sodium value is to be determined in said sodium image data set; and
providing said processor with reference image data also acquired using said sequence configured for sodium-23 imaging sequence and, in said processor, determining said sodium value by comparing image data in said at least one sodium image data set, that represent a region of interest within said target region, with said reference image data.

2. A method as claimed in claim 1 comprising generating said reference image data by placing a phantom in said magnetic resonance data acquisition unit and acquiring said reference image data from said phantom together with acquisition of said at least one sodium image data set from said target region.

3. A method as claimed in claim 2 comprising integrating said phantom with a local coil and using said local integrated with said phantom to acquire said at least one sodium image data set.

4. A method as claimed in claim 2 comprising placing said phantom in said magnetic resonance data acquisition unit immediately adjacent to said target region when said at least one sodium image data set and said reference image data are being acquired.

5. A method as claimed in claim 4 comprising identifying skin in said target region, as said region of interest, from a position of the skin in said target region relative to said phantom.

6. A method as claimed in claim 2 comprising providing said phantom with a plurality of containers or receptacles respectively for materials having respectively different N+ content.

7. A method as claimed in claim 6 comprising filling at least one of said containers or receptacles with a material selected from the group consisting of a sodium chloride solution and NaCl in 5% agarose.

8. A method as claimed in claim 6 comprising providing said phantom with at least four of said containers or receptacles, and respectively filling said at least four containers or receptacles with at least four different materials having respective Na+ contents that, in succession, equidistantly differ from each other with regard to said Na+ content.

9. A method as claimed in claim 8 comprising employing, as said at least four materials, materials respectively with 10, 20, 30 and 40 mM NaCl.

10. A method as claimed in claim 8 comprising employing, as said at least four materials, materials respectively with 0, 20, 40 and 60 mM NaCl.

11. A method as claimed in claim 6 comprising, in said processor, identifying respective positions of the respective materials in the respective containers or receptacles by subjecting said reference image data to a segmentation algorithm in said processor.

12. A method as claimed in claim 1 comprising operating said magnetic resonance data acquisition unit to acquire said at least one sodium image data set and said reference image data with a basic magnetic field strength of at least 3 Tesla.

13. A method as claimed in claim 12 comprising operating said magnetic resonance data acquisition unit to acquire said at least one sodium image data set and said reference image data with a basic magnetic field strength of at least 7 Tesla.

14. A method as claimed in claim 1 comprising operating said magnetic resonance data acquisition unit with a gradient echo sequence for said sodium-23 imaging.

15. A method as claimed in claim 14 comprising employing a gradient echo sequence having an echo time of at least 2 ms.

16. A method as claimed in claim 14 comprising operating said magnetic resonance data acquisition unit with a gradient echo sequence comprising more than one echo.

17. A method as claimed in claim 16 comprising employing a gradient echo sequence with up to twelve echoes.

18. A method as claimed in claim 1 comprising acquiring said at least one sodium image data set with a pulse sequence comprising echo times that are shorter than 1 ms.

19. A method as claimed in claim 18 comprising employing a radial sequence as said pulse sequence.

20. A method as claimed in claim 1 wherein said magnetic resonance data acquisition unit generates a basic magnetic field (B1) that exhibits B1 inhomogeneities, and, in said processor, implementing a correction of at least said at least one sodium image data set to correct said B1 inhomogeneities.

21. A method as claimed in claim 20 comprising acquiring said at least one sodium image data set by operating said magnetic resonance data acquisition unit with a B1 field strength of at least 7 Tesla and using a local coil matched to sodium-23 imaging.

22. A method as claimed in claim 20 comprising operating said magnetic resonance data acquisition unit to acquire a correction image data set of a subject having a homogenous Na+ content that is located at the position of the target region using said sodium-23 imaging sequence, and providing said correction image data to said processor for implementing said correction of Bi inhomogeneities.

23. A method as claimed in claim 22 wherein each of said correction image data set and said at least one sodium image data set is comprised of image points, and implementing said correction of said B1 inhomogeneities in said processor image point-by-image point.

24. A method as claimed in claim 1 comprising operating said magnetic resonance data acquisition unit using a hydrogen imaging sequence to acquire at least one anatomy image data set of said target region, that depicts anatomy in said target region, with the target region being in a same position in said magnetic resonance data acquisition unit as when said at least one sodium image data set is acquired, and identifying said region of interest in said processor by segmenting said region of interest from said anatomy image data set and transferring the segemented region of interest to said at least one sodium image data set.

25. A method as claimed in claim 24 comprising segmenting regions in said anatomy image data set selected from the group consisting of aqueous regions and blood vessel regions that include visible blood vessels, and excluding said selected regions from said at least one sodium image data set when determining said sodium value.

26. A method as claimed in claim 24 comprising segmenting skin from said anatomy image data set as said region of interest by delineating said skin from a region comprising air using a threshold.

27. A method as claimed in claim 26 comprising using a threshold value that represents twice a value of background noise.

28. A method as claimed in claim 1 comprising determining said sodium value in said processor using a linear trend analysis based on reference image data for at least two different sodium contents.

29. A method as claimed in claim 1 comprising determining said sodium value by identifying a disruption in a distribution of Na+ in said region of interest.

30. A method as claimed in claim 1 comprising determining said sodium value in said processor by identifying a disruption of an absolute content of Na+ in said region of interest.

31. A method as claimed in claim 1 comprising operating said magnetic resonance data acquisition unit to acquire a plurality of sodium image data sets of said target region at successive times and determining at least one sodium value for each of said sodium image data sets, and, in said processor, generating a curve of the respective sodium values with respect to time.

32. A local coil assembly to acquire a sodium image data set in a magnetic resonance data acquisition unit, comprising:
a local coil configured to detect magnetic resonance signals original from excited nuclear spins in an examination subject located in a magnetic resonance data acquisition unit;
a phantom comprising a plurality of containers or receptacles respectively containing materials each having a predetermined Na+ content; and
said local coil and said phantom being each mechanically shaped in order to mechanically integrate said phantom with said local coil.

33. A local coil assembly as claimed in claim 32 wherein said local coil comprises at least two coil elements each configured to acquire sodium-23 magnetic resonance signals.

34. A local coil assembly as claimed in claim 32 comprising a covering layer over said containers or receptacles having a thickness of less than 1 millimeter.

35. A local coil assembly as claimed in claim 34 wherein said covering layer is selected from the group consisting of membranes and films.
